(19) United States
(12) Patent Application Publication
(10) Pub. No.: US 2009/0061508 A1
(43) Pub. Date: Mar. 5, 2009

(54) Laminar flow reactor

(75) Inventors: Meir Israelowitz, Toronto (CA); Herbert Peter von Schroeder, Toronto (CA); Syed Rizvi, Lake Mary, FL (US); Chris Holm, Wuppertal (DE); Christoph Gille, Hohen Peuendorf (DE)

Correspondence Address: Herbert P. von Schroeder, 191 Ellis Avenue, Toronto, ON, M6S 2X4, CA

(73) Assignee: BIOMIMETICS TECHNOLOGIES INC, Toronto (CA)

(21) Appl. No.: 11/895,645

(22) Filed: Aug. 27, 2007

(51) Int. Cl. C12M 1/00

(52) U.S. Cl. 435/289.1

(57) ABSTRACT

A Laminar Flow reactor for large scale culturing or packaging of cell suspensions, three-dimensional tissue and other biological systems is disclosed. The Laminar Flow reactor consists of an upper housing and a lower housing. The housings are interconnected by a plurality of connectors and a plurality of rigid spacers, an inlet fluid, an outlet fluid, a fluid reservoir, and a means for transporting fluid within the system. During treatment, liquid media is transported from the fluid reservoir to the inlet manifold, which will in turn evenly distribute the media to each of the connected connectors and internal culture pockets. Laminar flow through the device is maintained by the cylindrical shape of the device and by bypasses. An outlet fluid manifold is also provided to ensure that each treatment chamber is evenly filled and to ensure that any air bubbles formed during treatment are removed from the treatment chambers.

### RELATED APPLICATION INFORMATION

This application claims priority to U.S. Patent Application No. 60/838,494, filed August, 2006, which is hereby incorporated by references in its entirety.

### FIELD OF INVENTION

The reactor of the invention relates to explore the characteristics of biological materials and allows for the culture and growth of such materials. These characteristics are important for tissue engineering applications, which include but are not limited to replacement of tissues such as skin and bone or replacement of organs or parts thereof. Existing bioreactors or fermentors have the capacity for growing small quantities of tissue. A limitation of fermentor devices is their design that allows for, or creates shear forces which contribute to high mortality of the cells in culture. Many existing bioreactors require enzyme kinetics, membrane exchange or use rotational designs to mimic a micro-gravity environment.

By definition, a reactor is described as a mass-transfer of rate controls and of rate of reaction. The intrinsic catalyzed reaction rate must establish the rate expression. The design must either incorporate both the reaction rate and the mass-transfer rate or neglect the reaction rate with justification by comparison with the mass-transfer rate.

Reactor designs are categorized for industrial and small scale. The following bioreactors models bioreactors are being used today:

Rotational reactors use continuous spin to mimic a microgravity environment. The spin reduces the hydrodynamic shear ten-fold. The reduction of shear is important to maintain the cell structure and integrity. Another property in the design is the increased mass transfer to support large aggregates. A limitation with this design is that it cannot be used to adequately control the growth of large amounts of cells in a scaffold.

Stirred methods, or suspension bioreactors, are commonly used in life sciences and chemical research. The major limitation of these types of bioreactors is that the design cannot be transferred to industrial sizes. By scaling-up such bioreactors, the resulting increase in the shear forces limits the sizes of cultures and hence limits the practical applications.

Industrial scale bioreactors include, for example, the Air Lift Bioreactor. The Air Lift reactor is a low energy input, high yield alternative to stirred tank bioreactors. Its low shear action allows for good oxygen transfer, but the tissue culture sizes and volumes cannot be adequately controlled.

The terms reactors or fermentors are commonly used in the trade because they describe a control mass-transfer and a control rate of reaction in the mass-transfer. Typical bioreactors or fermentors of this type are shown, for example, in the following United States of America patents:

| | |
|---|---|
| 6,752,921 | Kulick |
| 6,680,166 | Mullon, et al |
| 6,670,173 | Schels, et al |
| 6,670,169 | Schob |
| 6,632,658 | Schob |
| 6,632,657 | Kislyh |
| 6,589,780 | Banerjee, et al |
| 6,582,955 | Martinez, et al |
| 6,582,596 | Mao |
| 6,566,126 | Cadwell |
| 6,933,144 | Cadwell |
| 6,423,229 | Mao |
| 6,228,607 | Corcho-Sanchez, et al |
| 6,844,187 | Wechler, et al |
| 6,228,607 | Kersten |
| 6,168,949 | Rubenberger |
| 6,214,617 | Herman |
| 6,133,019 | Herman |
| 6,867,040 | Helmstetter, et al |
| 5,998,184 | Shi |
| 5,882,918 | Mausli |
| 5,846,816 | Forth |
| 5,763,267 | Kurjan, et al |
| 5,705,390 | Kadouri, et al |
| 6,680,166 | Mullon, et al |
| 5,622,819 | Herman |
| 5,605,835 | Hu, et al |
| 5,587,298 | Horigane |
| 5,565,361 | Musakis, et al |
| 6,001,642 | Tsao |
| 5,563,068 | Zhang |
| 5,556,765 | Dedolph |
| 5,527,467 | Ofsthum |
| 5,955,353 | Amiot |
| 5,531,897 | Stormo |
| D370,263 | Falkenberg, et al |
| 5,494,577 | Rekers |
| 5,494,574 | Unterman |
| 5,476,783 | Mutsakis, et al |
| 5,376,548 | Matsuo, et al |
| 5,320,963 | Knaack, et al |
| 5,256,570 | Clyde |
| 5,158,593 | Delima |
| 5,155,219 | Kim, et al |
| 5,155,035 | Schwarz |
| 5,308,764 | Goodwin |
| 5,137,828 | Robison |
| 5,057,428 | Mizutani |
| 4,937,196 | Wrasidlo, et al |
| 4,931,401 | Safi |
| 4,894,342 | Guinn, et al |
| 4,892,818 | Ramp |
| 4,889,812 | Guinn, et al |
| 4,833,089 | Kojima, et al |
| 4,833,083 | Saxena |
| 4,833,081 | Walker |
| 4,654,308 | Safi |
| 4,649,117 | Familletti |
| 4,636,473 | Kleinstreuer |
| 4,032,407 | Scott, et al |
| 6,083,8494 | Israelowitz, et al |

### REFERENCES

All the publications and patents mention herein, are hereby incorporated by reference in their entirely as if each individual publication or patent was specifically and individually indicated to be incorporated by reference. In case of conflict, the present application, including any definition herein, will control.

### EQUIVALENTS

While the specific embodiments have been discussed, the above specification is illustrative and not restrictive. Many variations will become apparent to those skilled in the art upon review of this specification. The full scope of the disclosure should be determined by reference of the claims, along with their full scope of equivalents, and specifications, along such variations.

### BACKGROUND OF THE INVENTION

It is well known in the art that the growth and production of biologicals requires medium for cell and tissue nourishment. Exchange of medium is vital to supply such nourishment as well as remove toxins, metabolic end-products and waste. The exchange and flow of medium is optimally achieved by a laminar flow over and around the cells and through supportive scaffolding. The laminar flow reduces turbulence and shear forces that otherwise impede the growth of the biological material. Laminar flow can also allow for the rapid exchange of nutrients and waste products.

Our design is a non-rotating bioreactor capable of growing large volumes or amounts of tissue by controlling the shear forces. The shear forces are reduced by the shape of the bioreactor and by maintaining laminar flow of the medium. Laminar flow is achieved by a pump and pressure sensors that control and steer the flow of medium.

The design allows for the placement of a scaffold in the middle of the device. The purpose of the scaffold is to support cell growth and provide structural integrity of the tissue or organ. The cells can proliferate and differentiate within or on the scaffold. Providing optimal delivery of medium and optimal shear forces is vital to reduce cell death and to increase the rate of adaptation for the cells in the scaffold.

### SUMMARY OF THE INVENTION

The invention brings a new kind of control to optimize laminar flow of fluid and thereby stimulate cell growth in three dimensions. Fluid flow is achieved through the cellular biological sample and through a bypass mechanism with variables openings. By optimizing laminar flow, turbulence is reduced. For this purpose the reactor consists of a housing that includes a chamber for intake of biomaterial and variable bypasses around the chamber.

The flow through this reactor can be precisely controlled without influencing or controlling the pump, thus improving pump efficiency. Specials features of this bioreactor are the two rings around the chamber that have openings for directing the fluid flow through a bypass as needed to control the flow.

The reactor could be equipped with a handle on any one of the rings with an iris to open and close the openings to steer the flow; it could be moved by hand or motor and could be automated.

The reactor can contain pressure sensors to control the handle and iris and hence the fluid flow.

It is an advantage to have a window at the output of the chamber showing the kind of flow. The device can also verify or quantify the flow.

An advantage of the laminar flow bioreactor is the whole biomaterial will have access to medium and a result is the cells will growth in a 3-Dimensional growth.

A second advantage the flow produce buyoand force creating microgravity environment, which help in the initial cell development.

A third advantage is by having laminar flow the shear force will not destroy the cells.

### BRIEF DESCRIPTION OF THE DRAWINGS

FIG. 1 shows a longitudinal assembly of the reactor

FIG. 2 shows a longitudinal middle section of the assembly

FIG. 3 shows sections of longitudinal assembly in 3-Dimensions

FIG. 4 shows a longitudinal assembly in 3-Dimensions

FIG. 5 shows a 3-Dimensional assembly of the irises

FIG. 6 shows a 2-Dimensional flow of the reactor with the iris close

FIG. 7 shows a 2-Dimensional flow of the reactor with the iris open

FIG. 8 shows the reactor system assembly

### DESCRIPTION OF PREFERRED EMBODIMENT

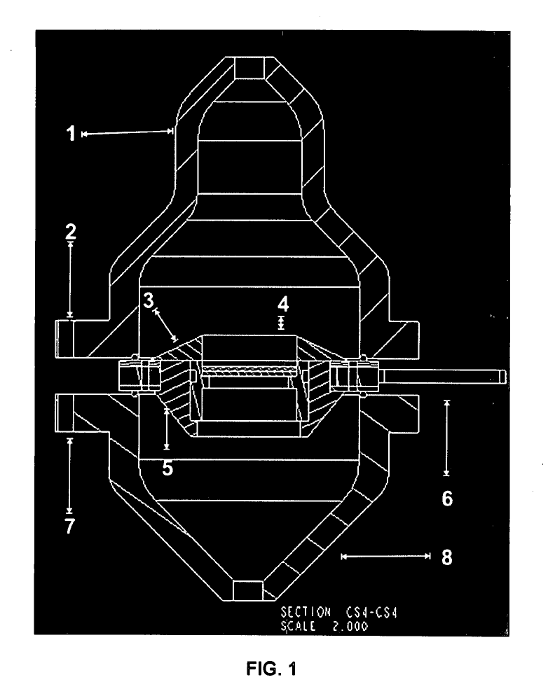

FIG. 1 Diagram of the bioreactor is in a cross section, including the assembly and all the connected parts. The UPPER_HOUSE (1) is connected with TOP_FLANGE (2). The SCAFFOLD_HOLDER (4) is in the middle, with the grid. The IRIS_HANDLE (5) is connected with the IRIS_CLAMP (3) and the LOCKRING (5). The LOWER_FLANGE (7) is connected with the LOWER_HOUSE (8).

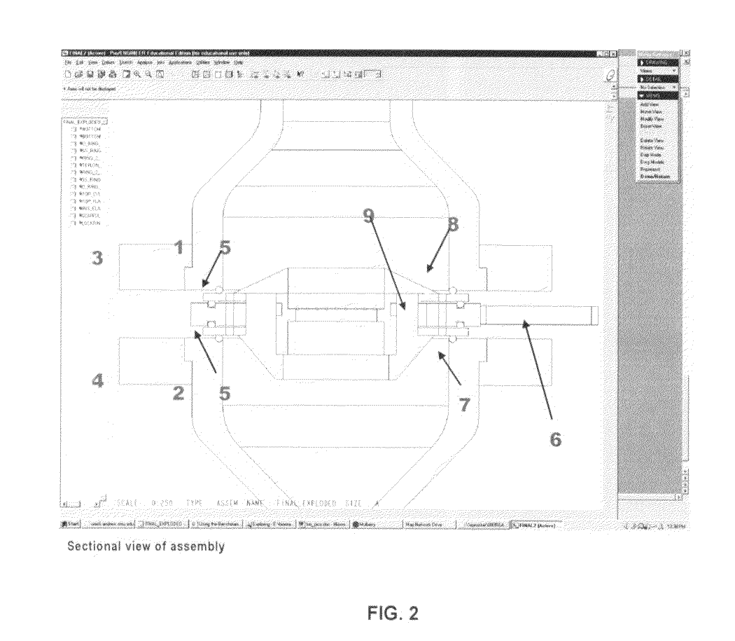

FIG. 2 Cross section of the reactor and the numbers indicate part in the cross section:

UPPER_HOUSE is show by (1), the LOWER_HOUSE is show by (2), the UPPER_HOUSE and the LOWER_HOUSE parts are the primary contributors to the inside shape of the bioreactor and the shape is important for the LAMINAR FLOW. The TOP_FLANGE (3) is a single piece part of the UPPER_HOUSE (1). The LOWER_FLANGE (4) is a single piece part of the LOWER HOUSE (2). The SS_RING (5) these two rings provide the sealing surface for both the top and bottom half of the assembly as well as either side of IRIS_HANDLE (6). A total of four o-rings interact with the two rings. The SS_RING (5) not only provide for the overflow system but also serve as the primary structural components that hold the IRIS_CLAMP (7) assembly as well as the scaffold holder and the scaffold itself. The flat area on the inside diameter of the part interfaces with the notch out of the protrusion of IRIS_CLAMP (7) to ensure that the both SS_RING (5) parts are correctly positioned relative to each other. When assembled, both instances of SS_RING (5) line up with each other. When IRIS_HANDLE (6) is rotated, one should be able to see through the overflow valves (all three layers). The IRIS_HANDLE (6) is the only moving part in the assembly, that which allows the size of the overflow valves to be modified. To maintain the sealing provide by the two grooves, one on each side, were added to seat two O_RINGS (not showing in this drawing). Between the SS_RING (5) and the SCAFFOLD_HOLDER (9) they are two grooves with the second set O_RINGS for sealing internal leakages.

The arm extending from the circular portion of the part functions to connect to the computer and controlled linear actuator to change the valving incrementally as conditions across the scaffold (bio-material) change. The IRIS_CLAMP (8) provides support for the scaffold basket as well as a surface and mass to clamp the whole valve/scaffold support subassembly together. The outer section of the IRIS_CLAMP (8) provides the contact surface for the bottom of the SS_RING (5)/IRIS_HANDLE (6) assembly (the top is provided by LOCKRING (7)) and the inner is a resting point for the scaffold holder. The flat is simply to keep the valving subassembly properly aligned. The LOCKRING (7) is simply the part that allows the valving subassembly to be held firmly together. To complete this assembly, Screws are put through LOCKRING (7) and well into IRIS_CLAMP (8). The shape of LOCKRING is nothing more than an attempt at making the fluid flow within the bioreactor more suitable. This shape can be modified to better suit the application.

SCAFFOLD_HOLDER (9) fit in the assembly is designed to be interchangeable.

The SCAFFOLD_HOLDER (9) is a cylinder with a tab around the circumference of the inside and a groove around the outside circumference. A number of small holes are drilled parallel to each other across the center of this groove and along the rim around the inside give the support for the scaffold.

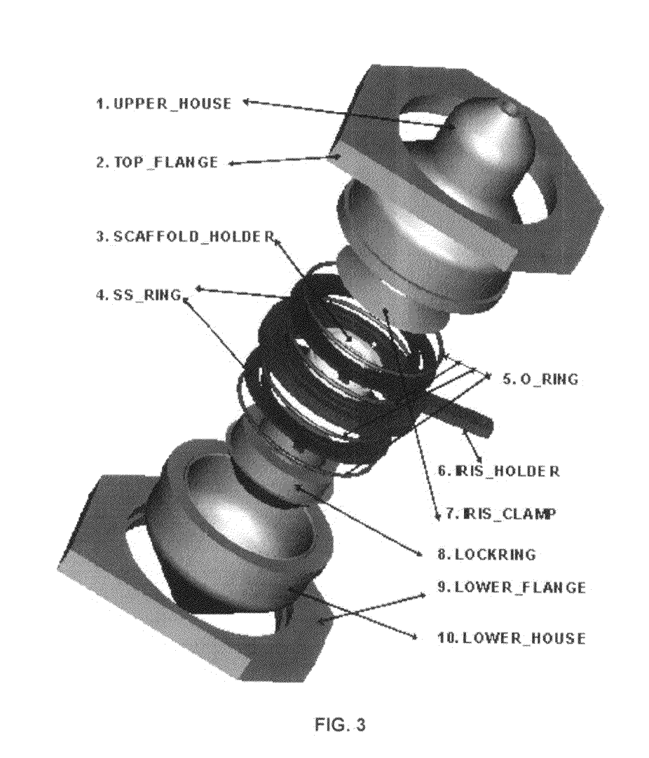

FIG. 3 3-Dimensional illustration of each section in the reactor where: the UPPER_HOUSE (1) is in place with the TOP_FLANGE (2). The SCAFFOLD_HOLDER (3) is located in the middle of the assembly. Two SS_RINGS (4) are holding the IRIS_HOLDER. Two set's of O_RINGS (5) to seal internal leak and the houses. The IRIS_HOLDER (6) is connected to the control box, moving the handle opens or closes the bypasses. The IRIS_CLAMP (7) holds the irises in place. The LOWER_HOUSE (8) in the end of the assembly is with the LOWER_FLANGE (9).

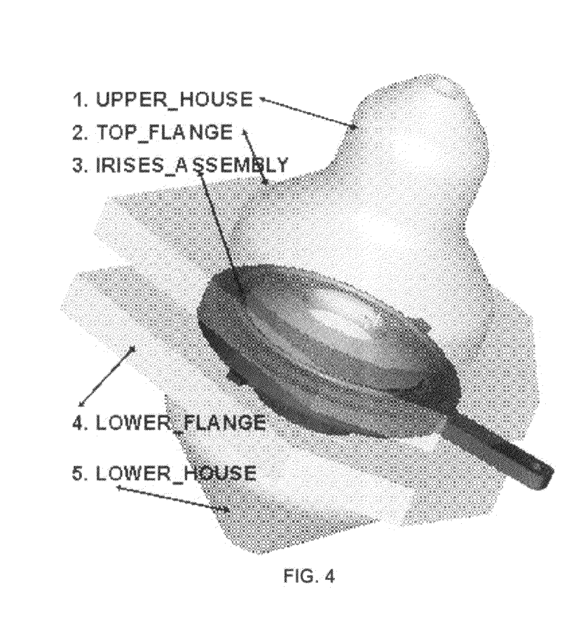

FIG. 4 3-Dimensional assembly of the reactor where: the UPPER_HOUSE (1) is with the TOP_FLANGE (2). The irises assembly is show in the middle (3). The LOWER_FLANGE (4) is with the LOWER_HOUSE (5).

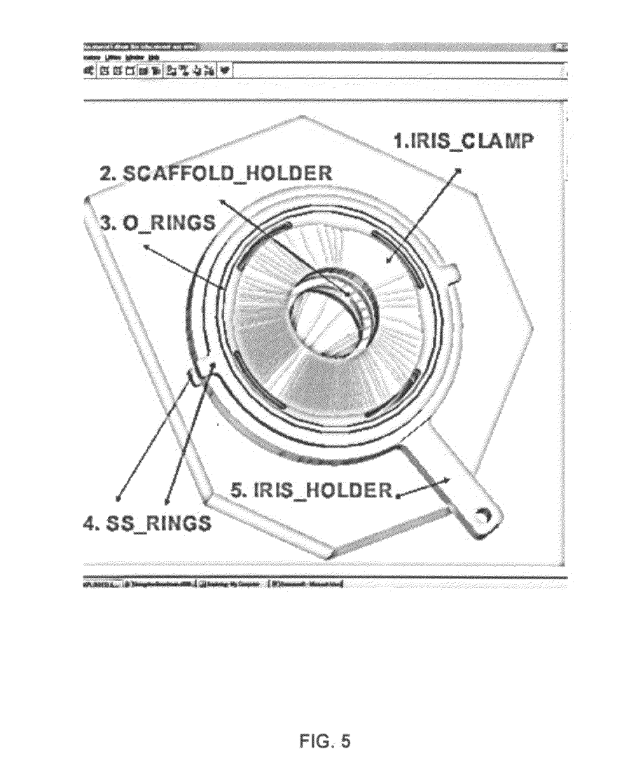

FIG. 5 Assembly of irises where: the IRIS_CLAMP (1) is the top of the assembly. In the middle is showing the SCAFFOLD_HOLDER (2). The O_RING (3) is over the SS_RING (4), to seal any internal leak. The SS_RING (4) is between the IRIS_HOLDER (5).

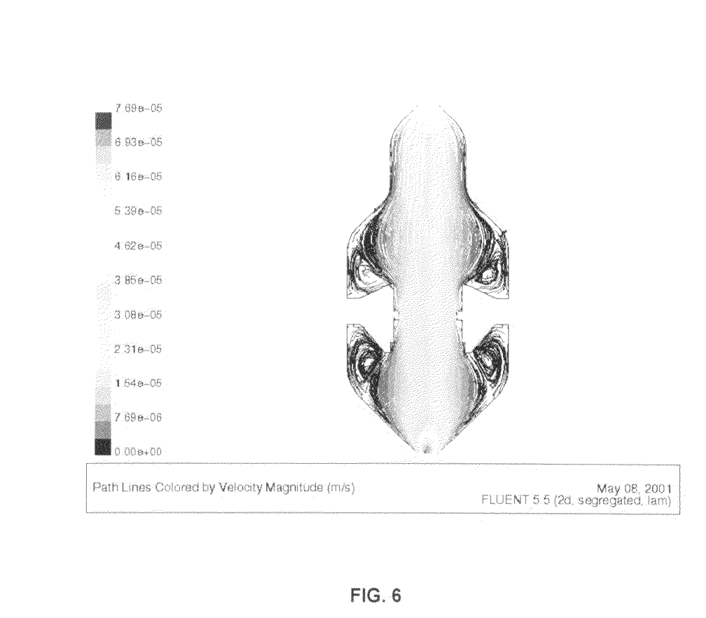

FIG. 6 Flow path lines with the irises closed.

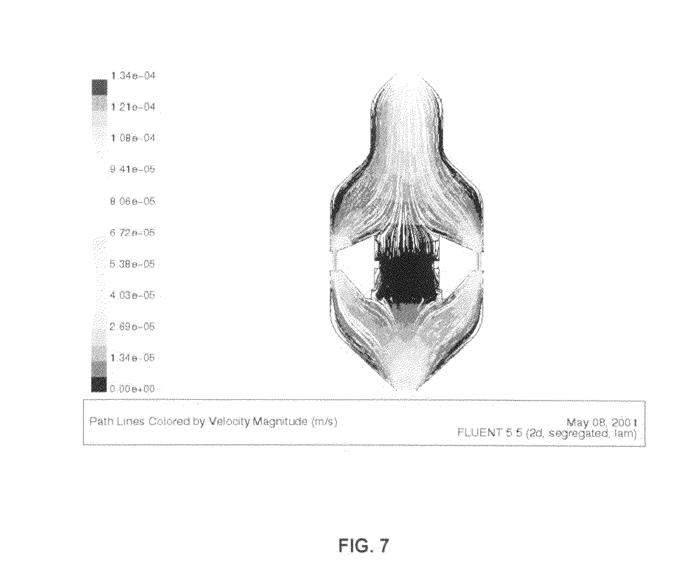

FIG. 7 Flow path lines with the irises open.

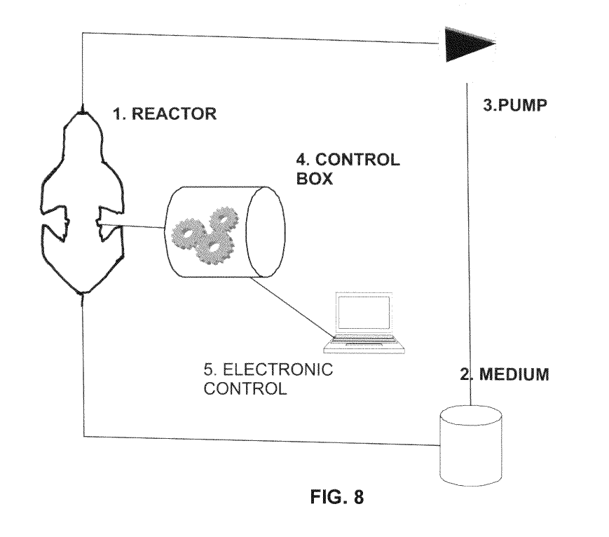

FIG. 8 Bioreactor (1) is connected to the cell medium (2). The cell medium is connected to pump (3). The reactor is connected to the control box (4) and control box is connected to the electronic control (5)

1. Laminar flow reactor for three dimensional cell growth consisting of a housing including a chamber for intake of bio-material and variable bypasses around the chamber.

2. Reactor according claim 1 is a combination of plurality of two houses and assembly of bypasses.

3. Reactor according claim 1 is a combination of plurality interlock bypasses and the bypasses is connected to the upper house.

4. Reactor according claim 1 is a combination of plurality interlock bypasses and the bypasses are connected to the lower house.

5. Reactor according claim 1 is a combination of plurality of bypasses and an integral part of the upper house.

6. Reactor according claim 1 is a combination of plurality of bypasses and an integral part of the lower house.

7. Reactor according claim 1 is a combination of plurality of bypasses and an integral part of the houses.

8. Reactor according claim 1 with two rings around the chamber having openings for steering the flow through the bypass.

9. Reactor according claim 1 with two rings around the chamber is a combination of plurality bypasses connected to the upper house.

10. Reactor according claim 1 with two rings around the chamber is a combination of plurality bypasses connected to the lower house.

11. Reactor according claim 1 with two rings around the chamber is a combination of plurality bypasses integral of the upper house.

12. Reactor according claim 1 with two rings around the chamber is a combination of plurality bypasses integral of the lower house.

13. Reactor according claim 1 with two rings around the chamber is a combination of plurality bypasses integral of the houses.

14. Reactor according to claim 1 with a handle on a ring with iris to open and close the openings to steering the flow.

15. Reactor according to claim 1 with a handle on a ring with iris to open and close the openings to steering the flow and plurality bypasses connected to the upper house.

16. Reactor according to claim 1 with a handle on a ring with iris to open and close the openings to steering the flow and plurality bypasses connected to the lower house.

17. Reactor according to claim 1 with a handle on a ring with iris to open and close the openings to steering the flow and plurality bypasses connected to the houses.

18. Reactor according to claim 1 with a handle on a ring with iris to open and close the openings to steering the flow combination of plurality bypasses integral of the upper house.

19. Reactor according to claim 1 with a handle on a ring with iris to open and close the openings to steering the flow combination of plurality bypasses integral of the lower house.

20. Reactor according to claim 1 with a handle on a ring with iris to open and close the openings to steering the flow combination of plurality bypasses integral of the houses.

21. Reactor according to claim 20 with sensors steering the motion of a motor moving the handle.

22. Reactor according to claim 20 with sensors steering the motion of a motor moving the handle and plurality bypasses connected to the lower upper house.

23. Reactor according to claim 20 with sensors steering the motion of a motor moving the handle and plurality bypasses connected to the lower house.

24. Reactor according to claim 20 with sensors steering the motion of a motor moving the handle and plurality bypasses connected to the houses.

25. Reactor according to claim 20 with sensors steering the motion of a motor moving the handle combination of plurality bypasses integral of the upper house.

26. Reactor according to claim 20 with sensors steering the motion of a motor moving the handle combination of plurality bypasses integral of the lower house.

27. Reactor according to claim 20 with sensors steering the motion of a motor moving the handle combination of plurality bypasses integral of the houses.

28. Reactor according to claim 1 with a chamber with a grid to support the bio-material.

29. Reactor according to claim 1 with a chamber with a grid to support the bio-material and plurality bypasses connected to the upper house.

30. Reactor according to claim 1 with a chamber with a grid to support the bio-material and plurality bypasses connected to the lower house.

31. Reactor according to claim 1 with a chamber with a grid to support the bio-material and plurality bypasses connected to the houses.

32. Reactor according to claim 1 with a chamber with a grid to support the bio-material combination of plurality bypasses integral of the upper house.

33. Reactor according to claim 1 with a chamber with a grid to support the bio-material and combination of plurality bypasses integral of the lower house.

34. Reactor according to claim 1 with a chamber with a grid to support the bio-material and combination of plurality bypasses integral of the houses.

35. Reactor according to claim 1 with a window at the output of the chamber showing the kind of flow and the plurality of bypasses connected to the house.

36. Reactor according to claim 1 with a window at the output of the chamber showing the kind of flow.

37. Reactor according to claim 1 with a window at the output of the chamber showing the kind of flow and the plurality of bypasses connected to the upper house.

38. Reactor according to claim 1 with a window at the output of the chamber showing the kind of flow and the plurality of bypasses connected to the lower house.

39. Reactor according to claim 1 with a window at the output of the chamber showing the kind of flow and combination of plurality bypasses integral of the upper house.

40. Reactor according to claim 1 with a window at the output of the chamber showing the kind of flow and combination of plurality bypasses integral of the lower house.

41. Reactor according to claim 1 with a window at the output of the chamber showing the kind of flow and combination of plurality bypasses integral of the houses.
